(19) United States
(12) Patent Application Publication
(10) Pub. No.: US 2014/0110349 A1
(43) Pub. Date: Apr. 24, 2014

(54) ASSEMBLY AND METHOD FOR FILTRATION

(71) Applicants: Joachim Bangert, Erlangen (DE); Katja Friedrich, Erlenbach a. Main (DE); Walter Gumbrecht, Herzogenaurach (DE); Karsten Hiltawsky, Schwerte (DE); Peter Paulicka, Roettenbach (DE); Manfred Stanzel, Berching (DE)

(72) Inventors: Joachim Bangert, Erlangen (DE); Katja Friedrich, Erlenbach a. Main (DE); Walter Gumbrecht, Herzogenaurach (DE); Karsten Hiltawsky, Schwerte (DE); Peter Paulicka, Roettenbach (DE); Manfred Stanzel, Berching (DE)

(73) Assignee: SIEMENS AKTIENGESELLSCHAFT, Munich (DE)

(21) Appl. No.: 14/118,728

(22) PCT Filed: Apr. 13, 2012
(86) PCT No.: PCT/EP2012/056804

§ 371 Date: Dec. 18, 2013

(30) Foreign Application Priority Data
May 20, 2011 (DE) 102011076228.0

(51) Int. Cl. A61M 1/34; B01D 61/18; B01D 61/22

(52) U.S. Cl. CPC A61M 1/34; B01D 61/22; B01D 61/18
USPC 210/741; 210/137; 210/104; 210/90

(57) ABSTRACT

An assembly and a method for filtration are disclosured, which are suitable in particular for the filtration of cells, for example tumor cells, from a sample, for example a blood sample. In the method, a pressure differential between the pressure upstream and the pressure downstream of a filter is determined; and the pressure differential between upstream and downstream of the filter is adjusted such that the pressure differential does not exceed a predetermined value.

### PRIORITY STATEMENT

This application is the national phase under 35 U.S.C. §371 of PCT International Application No. PCT/EP2012/056804 which has an International filing date of Apr. 13, 2012, which designated the United States of America and which claims priority to German patent application number DE 10 2011 076 228.0 filed May 20, 2011, the entire contents of each of which are hereby incorporated herein by reference.

### FIELD

At least one embodiment of the invention generally relates to an assembly and/or to a method for filtration which is suitable in particular for the filtration of cells, for example tumor cells, from a sample, for example a blood sample. In at least one embodiment of the method, a pressure differential between the pressure upstream and the pressure downstream of a filter is determined; and the pressure differential between upstream and downstream of the filter is adjusted such that the pressure differential does not exceed a predetermined value.

### BACKGROUND

The detection of circulating tumor cells (CTC) in the blood is of ever-increasing significance for the early recognition, diagnosis and therapeutic monitoring of cancer. Due to the low number of CTCs, which may be in the range of only 3-5 in a milliliter of blood, and due to the large background of leucocytes (6-10×106 per milliliter) a method must be chosen which is able to accumulate CTCs as selectively as possible or to display them in the presence of a large surplus of other blood cells.

A method for the detection of CTCs comprises the filtration of blood samples, wherein by way of corresponding pore sizes cells are selected by size and tumor cells can be isolated. A disadvantage of this method is that the cells are often damaged by the filtration process itself and can then only be used to a limited extent for further examinations.

“Dead-end filtration” using a partially permeable membrane forms the basis; the driving force is a pressure gradient. A feed is filtered through the membrane, wherein the liquid is able to permeate the membrane (permeate) and larger particles accumulate on the membrane as a filter cake (retentate).

Dead-end filtration gives rise to various problems:

A filter cake (top layer or fouling) accumulates on the membrane as a result of the permanent drainage of permeate (or a concentration gradient/concentration polarization) from the retentates. The filter cake increases filtration resistance and thereby the loss of pressure via the membrane.

Furthermore, the permeate flow declines increasingly as a result of the permanent flow of the feed. Purification stages (for example, back washing) produce an intermittent feed flow which may result in falls in production, the use of cleaning agents, and additional technical expenditure.

The changes in the filter cake make a calculation of the filter conditions almost impossible.

From a technical point of view, the following technical problems for filtration thereby stem from this: there are fluctuating pressure conditions, a fluctuating feed flow, and an unknown time until blockage. The filtration properties alter. Filtration applications in which linear filter behavior and reproducible results are necessary suffer from these problems. In addition, pressure-sensitive membranes, retentates or permeates are problematic if load limits are exceeded.

Until now efforts were made to avoid these disadvantages by pumping against the membrane with the minimum amount of pressure possible to minimize the compaction of the retained substances. The disadvantage of this is that a long period of filtration is necessary.

Removing the filter cake at regular intervals by way of back washing (pumping back medium which has already been separated) and chemical cleaning and thus regenerating the filter element is also known. Back washing produces a “saw-tooth pattern” in the feed flow. The disadvantage is that sensitive components of the filter cake may be damaged as a result.

### SUMMARY

At least one embodiment of the invention relates to a method and at least one embodiment of the invention relates to a device.

For filtration applications in which linear filter behavior and reproducible results are required, it is proposed that one or more of the following measures are taken:
Provision of a control by which the pressure differential or the feed flow can be adjusted to a constant value. The behavior of the filtration remains predictable.
optional increase in the usable filter area such that during the filtration process under consideration the retentate cannot lead to significantly altered filter properties (in extreme cases: blockage). Back washing and cleaning are rendered superfluous.
optional increase in the cavity or pore density such that during the filtration process under consideration the retentate cannot significantly lead to altered filter properties (in extreme cases: blockage). Back washing and cleaning are rendered superfluous.
optional simplification of the filter membrane such that the seepage flow equations can be modeled for control. It is therefore possible to largely exclude unexpected or unknown conditions of the filter cake.

In a filtration process according to at least one embodiment of the invention, a suspension is filtered through a filter, for example a filter membrane. In the process, permeate is pressed through the filter and retentate retained on the filter surface (or also in the pores and cavities of the filter). For the filtration process there is therefore a prevailing direction of flow for the permeate through the filter, making it possible to speak of an area upstream of the filter in which the retentate is retained, and an area downstream through which the permeate is pressed and, for example, where it can be collected. Regardless of this prevailing direction of flow, in exceptional cases the direction of flow can also be reversed, for example, when back washing the filter. The term “pressing through” also defines the prevailing direction of the pressure differential: the positive pressure differential between upstream and downstream. In the aforementioned exceptional case, if the pressure differential were negative, according to common parlance it could be described as suction.

At least one embodiment of the invention relates to a method for filtration of a suspension, comprising:

supply of the suspension to a filter;

determination of a pressure differential between the pressure upstream and downstream of the filter; and

adjustment of a pressure differential upstream compared with downstream of the filter and as a result pressing of permeate through the filter, wherein the pressure differential does not exceed a predetermined value.

Furthermore, at least one embodiment of the invention relates to a device for the performance of the method according to at least one embodiment of the invention, comprising:

a filter;

a liquid reservoir upstream of the filter;

a liquid reservoir downstream of the filter;

means of determining a pressure differential of the pressure upstream and downstream of the filter; and

means of adjusting a pressure differential upstream compared with downstream of the filter and as a result pressing of permeate through the filter, wherein the pressure differential does not exceed a predetermined value.

### BRIEF DESCRIPTION OF THE DRAWINGS

The invention is explained by way of example using the attached drawings and the following examples. The drawings show:

FIG. 1 a schematic representation of a filtration process;

FIG. 2 a schematic representation of a filtration device;

FIG. 3 a schematic representation of a control for performance of the method according to an embodiment of the invention.

### DETAILED DESCRIPTION OF THE EXAMPLE EMBODIMENTS

In a filtration process according to at least one embodiment of the invention, a suspension is filtered through a filter, for example a filter membrane. In the process, permeate is pressed through the filter and retentate retained on the filter surface (or also in the pores and cavities of the filter). For the filtration process there is therefore a prevailing direction of flow for the permeate through the filter, making it possible to speak of an area upstream of the filter in which the retentate is retained, and an area downstream through which the permeate is pressed and, for example, where it can be collected. Regardless of this prevailing direction of flow, in exceptional cases the direction of flow can also be reversed, for example, when back washing the filter. The term “pressing through” also defines the prevailing direction of the pressure differential: the positive pressure differential between upstream and downstream. In the aforementioned exceptional case, if the pressure differential were negative, according to common parlance it could be described as suction.

In order to press the permeate through the filter a pressure differential can be generated, wherein there is then a higher pressure upstream of the filter than downstream. This can be achieved by the application of overpressure upstream of the filter, application of underpressure downstream or a combination of the two. In order to stop (to reduce to zero) the permeate flow through the filter, a pressure differential of zero can be set. This is regardless of the orientation of the filter in the area. In the special event that the direction of flow runs vertically on the filter or a vertical component (in other words, in the direction of or contrary to the force of gravity), in addition it must be taken into consideration that the column of water on the filter contributes to the pressure differential.

For some applications of the method according to at least one embodiment of the invention, it is preferable that the direction of flow of filtration on the filter runs essentially in the direction of the force of gravity. As a result, retained retentate comes to lie on the surface of the filter which, for example, enables easy further processing of the retentate.

For certain applications it may be preferable to filter contrary to the force of gravity and not in the direction of the force of gravity, for example if the retentate floats.

At least one embodiment of the invention relates to a method for filtration of a suspension, comprising:

supply of the suspension to a filter;

determination of a pressure differential between the pressure upstream and downstream of the filter; and

adjustment of a pressure differential upstream compared with downstream of the filter and as a result pressing of permeate through the filter, wherein the pressure differential does not exceed a predetermined value.

A suspension is a liquid which contains solids suspended therein which are to be filtered.

According to one embodiment of the invention, it is preferable that the pressure differential does not exceed a value of 50 mbar, preferably 10 mbar. In some cases an upper limit for the pressure differential of 5 mbar, 1 mbar or less may be preferred.

According to one embodiment of the invention, it is preferable that when selecting the predetermined value of the pressure differential the pressure through the column of water is also taken into consideration and wherein a 1 cm column of water corresponds to approx. 1 mbar. The column of water corresponds to the filling level of the suspension above the filter in an essentially horizontal arrangement of the filter, wherein filtration takes place from top to bottom.

In addition to the position relative to the force of gravity, the selection of the predetermined value of the pressure differential also depends on several factors:

the pressure sensitivity of the retentate,

in the case of a vertical direction of flow through the filter, on the level of the column of water above the filter,

the nature of the filter, in particular pore size and pore density: for filters with many pores and/or large pores, a smaller relative pressure differential is necessary in order to filter the same volume in the same time than in the case of filtration with smaller or fewer pores,

the surface tension of the suspension

the viscosity of the suspension

According to one embodiment of the invention, it is preferable that when adding the suspension, the level of the column of water above the filter is monitored and limited to a predetermined fraction, for example ½, ¼, or preferably to 1/10 of the pressure differential. A fraction of 1/10 would correspond to, for example, a contribution of 1 mbar by the column of water at a pressure differential of 10 mbar, which corresponds to a filling level of 1 cm above the filter, and a contribution, for example, by underpressure downstream of the filter of 9 mbar.

According to one embodiment of the invention, it is preferable that overpressure is applied upstream of the filter.

According to one embodiment of the invention, it is preferable that underpressure is applied downstream of the filter.

According to one embodiment of the invention, it is preferable that the adjustment of the pressure differential by regulation to a constant value or to a value which is within a predetermined range takes place while permeate is being pressed through the filter.

This can be guaranteed by a corresponding simple control loop. This may comprise a differential pressure regulator. The differential pressure regulator may, for example, be designed as a valve which can selectively open and close a connection to an overpressure reservoir or an underpressure reservoir.

According to one embodiment of the invention, it is preferable that the volume of permeate which has passed through the filter is determined. For this purpose it is possible to measure the feed, to measure the filling level above the filter and to measure a filling level or a gas volume displaced by permeate downstream of the filter in a collection container.

In particular, according to one embodiment of the invention it is preferable to determine the volume of the permeate which has passed through the filter using a gas volume supplied when applying overpressure or using a gas volume discharged when applying underpressure.

According to one embodiment of the invention, it is preferable that the suspension is a suspension of cells in an aqueous solution. The method according to at least one embodiment of the invention is suitable in particular for the filtration and examination of circulating tumor cells (CTC) in liquid samples, for example blood samples. It is possible to filter the cells so carefully that functional tests can still be performed on the cells retained on the filter because the cells can be kept alive.

According to one embodiment of the invention, it is preferable that the filter has a membrane with pores, the direction of the pores of which runs exclusively vertically to the surface of the membrane.

According to one embodiment of the invention, it is preferable that the membrane is a track etched membrane made of polycarbonate.

In principle, all kinds of known membranes can be used for the filter (for example, flat membrane, filter bag (=second-level surface), hollow fiber application). On account of the easily predictable behavior and the great simplification of the seepage flow equations, a flat membrane with one or more of the following properties is particularly advantageous:

pore direction vertical to the surface (for example, as a result of limited membrane thickness relative to the pore size)

pore structure with few diameter errors

number and thickness of pores sufficiently great to avoid any significant alteration of the properties by the retentate

media and materials for inert use

possibly suitable for further processing of the retentate.

An example of such a membrane is a track etched membrane made of polycarbonate or of the COC with the commercial designation TOPAS (trade name).

According to one embodiment of the invention, the method comprises the additional step of the adjustment of a pressure differential for a predetermined period, which is selected such that during this period no permeate is pressed through the membrane.

According to one embodiment of the invention, the method comprises the additional step of the adjustment of overpressure downstream of the filter for a predetermined period, wherein the overpressure is at least as high as the pressure of the column of water above the filter. As a result, the retentate can be kept permanently covered in liquid. For the filtration of cells, it is possible to incubate these in various process liquids (for example, wash buffer, fixation buffer, permeabilization buffer, staining solutions, etc.). The filter is closed by way of corresponding overpressure “from below” as this overpressure counteracts the column of water.

According to one embodiment of the invention, it is preferable that the adjustment of a pressure differential takes place by means of a control. A control comprises the determination of a controlled variable (actual value), a comparison with a reference variable (target value) and the adjustment of the controlled variable to the reference variable.

Furthermore, at least one embodiment of the invention relates to a device for the performance of the method according to at least one embodiment of the invention, comprising:

a filter;

a liquid reservoir upstream of the filter;

a liquid reservoir downstream of the filter;

means of determining a pressure differential of the pressure upstream and downstream of the filter; and

means of adjusting a pressure differential upstream compared with downstream of the filter and as a result pressing of permeate through the filter, wherein the pressure differential does not exceed a predetermined value.

According to one embodiment of the invention, it is preferable that the device for determining a pressure differential comprise a differential pressure sensor.

According to one embodiment of the invention, in addition the device comprises a device for determining the filling level of a liquid above the filter.

According to one embodiment of the invention, in addition the device comprises a control for setting the pressure differential to a constant value or within a predetermined range of values while the permeate is being pressed through the filter.

According to one embodiment of the invention, it is preferable that a control system is available for the adjustment of overpressure downstream of the filter for a predetermined period, wherein the overpressure is at least as high as the pressure of the column of water above the filter.

According to one embodiment of the invention, in addition the device comprises means for determining the volume of the permeate pressed through the filter.

According to one embodiment of the invention, it is preferable that the filter has a membrane with pores, the direction of the pores of which runs essentially vertically to the surface of the membrane.

According to one embodiment of the invention, it is preferable that the membrane is a track etched membrane made of polycarbonate.

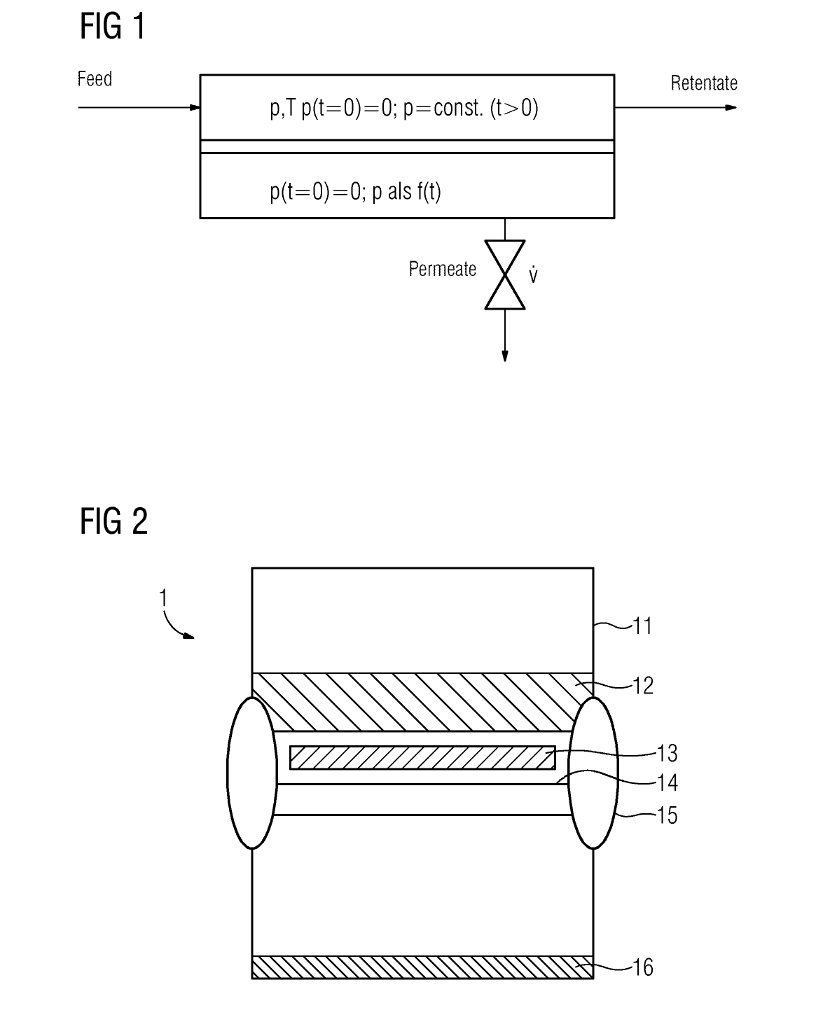

FIG. 1 shows a schematic representation of a filtration process, wherein a feed is routed via a filter, wherein a permeate runs through the filter and a retentate is retained.

FIG. 2 is a schematic representation of a filtration device 1 with a funnel or feed 11. The feed flow 12 is fed through a filtration device with a filter membrane 14 and retains a retentate (so-called filter cake) 13. Seals 15 create a leak-tight connection between the funnel 11 and the membrane 14 so that, for example, overpressure can be built up. The permeate 16 is collected in a permeate container (collection container). The collection container can also be arranged in a leak-tight connection with the membrane 14 so that, for example, underpressure can be built up.

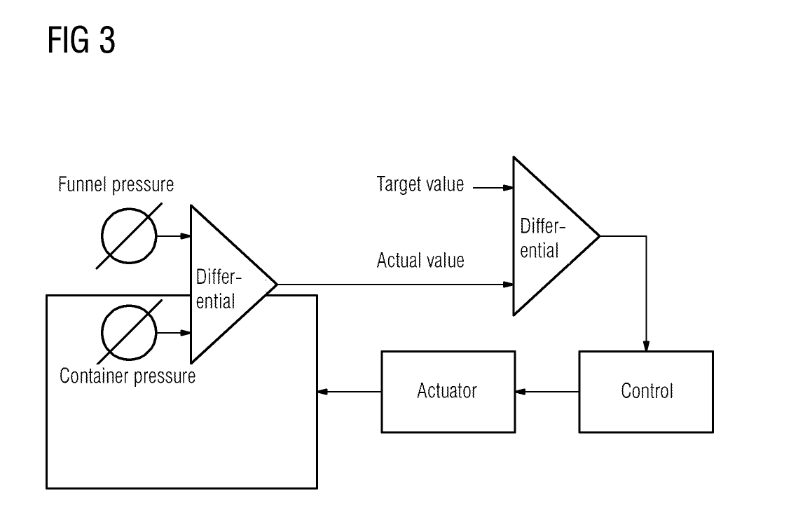

FIG. 3 is a schematic representation of an exemplary control for performance of the method according to the invention. A pressure differential is measured between the funnel (feed) and the collection container (drain) via a differential pressure sensor and compared with a target value. A control unit adjusts underpressure in the collecting container (“container”) accordingly to ensure that the target value is observed.

The technical design of the driving force can be realized by means of pressure on the feed flow, by means of suction on the permeate or a combination. The embodiment described below is particularly advantageous:

Upstream of the filter there is normal pressure, a reservoir in front of the filter can be filled as required. The working air underpressure is applied in a collection container downstream of the filter. This causes the air to be sucked through the membrane onto the medium (the suspension for filtration). The resulting permeate then permeates through the filter. The permeate detaches itself from the filter at appropriate drainage points and runs into the collection container. The displaced air volume can be evaluated for further information (for example, feed flow determination).

This embodiment offers several advantages: there are no mechanical shearing stresses on the permeate. There is a minimum risk of contamination, the pressure stage (for example, a pump) does not come into contact with the permeate, the permeate does not come into contact with the pump components. The permeate remains in the container and can be further processed. In principle, the method can be used in any location.

In a first arrangement the technical design of this embodiment comprises a holding device for the filter which is geometrically aligned to the membrane, to the flow conditions and to the filling technology.

Preferably, additional microfluidic structure is available to optimize contact surfaces for reactions, evaporation areas, etc.

Preferably, the holding device for the filter is easy to clean or is designed as an economical disposable item, in combination with the filter itself as an option. Preferably the membrane of the filter rests on numerous, but small supporting points of the holding device.

The permeate can collect in a channel structure of the holding device. Drainage holes are provided in the holding device such that the air underpressure cannot escape through the membrane but only takes effect on the permeate.

Preferably drainage aids are provided on the drainage holes (for example, as collection ducts or guide tubes).

The handling properties must be ensured for the ongoing use of the retentate. Seals are preferably provided as standard seals (for example, O-ring seals), for example with preloading (by way of tension spring pressure, weight, etc.).

The collection container requires leak tightness and adequate compressive strength. Connections for filter/membrane and air pressure can be provided on it. The collection container is preferably easy to clean or can be an economical disposable item; the handling properties must be ensured for the ongoing evaluation of the permeate. A completely pre-assembled “reservoir” structure, consisting of the aforementioned components as a click kit, seems attractive.

It is preferable to limit the filling level above the filter such that the additional pressure as a result of gravitational force can be disregarded. Additional pressures on the feed flow and the retentate (for example, cells) by the resulting column of liquid are thereby minimized. A restriction to 1/10 of the pressure differential seems reasonable. For example, for 10 mbar a column of water of 1 mbar (=1 cm) must not be exceeded.

According to an alternative second arrangement, a reservoir for the suspension for filtration is provided in front of the filter and can be put under a defined (over)pressure similar, for example, to an injection. The reservoir can simply be set to air pressure (“open”). The defined pressure can be applied as a combination of volume changes (injection principle) and applied pressure (gas, liquids). This arrangement has similar advantages to the aforementioned first arrangement.

A feed flow and pressure control is required for filtration:

This is made possible, for example, by the determination of the pressure differential on the membrane (sensor diameter); the position of the sensors in the reservoir or in the feed or in the airflow-protected external space, for example by way of a differential pressure sensor.

For the simple arrangements intended, a proportional controller with an actuator for pressure adjustment including a source for pressure, in general overpressure and underpressure, is sufficient. This is shown in exemplary and schematic form in FIG. 3.

Furthermore, a measurement of the filtrate flow can be provided by way of permeate-volume determination from the controller error. The minimization of stress on the membrane and on the filtration material (for example, cells) is guaranteed by adjustable specifications of pressure and feed flow, for example for acceleration. By adjusting the pressure differential it is possible to stop the feed flow completely, wherein the adjustment compensates for influences caused by capillary effects, the force of gravity, etc. For example, this permits the action of reagents on the retentate, for example the staining of cells or their fixation by means of fixation reagents such as formaldehyde, etc. In the process, it is also possible to take into account and regulate capillary forces, for example due to the force of gravity, vapor pressure from the container, thermal expansion, etc.

One possibility for adjustment of the pressure differential is the provision of an air reservoir of suitable volume, and defined underpressure. The pressure differential is adjusted by calculating the volume of air for setting the desired pressure differential, using the valve opening time, the valve resistance and the pressure differential (target pressure minus air reservoir pressure).

An advantageous and tried and tested possibility for adjusting the working pressure is the provision of sufficiently resilient overpressure and underpressure devices from which the working pressure is removed by means of correspondingly controlled valves. In the first valve the overpressure or underpressure supply is selected, in a second valve a certain volume of air is transferred between the container and the supply by way of keying-in. This produces a temporal average which produces the working pressure.

In principle, all kinds of known filters or filter membranes can be used for filtration for the invention (for example flat membrane, coffee filter bag (=second-level surface), hollow fiber application). On account of the easily predictable behavior and the great simplification of the seepage flow equations, a flat membrane with the following properties is particularly advantageous:

pore direction vertical to the surface (for example, as a result of limited membrane thickness relative to the pore size),

pore structure with few diameter errors,

number and thickness of pores sufficiently great to avoid any significant alteration in properties as a result of the retentate,

media and materials for inert use, possibly suitable for further processing of the retentate.

For example: a track etched membrane made of polycarbonate or of TOPAS (trade name).

Possible applications of embodiments of the invention comprise cell separation, for example for “CTC” (circulating) tumor cells in the blood, tumor cells/urothelial cells in the urine, epithelial cells in the sputum, etc.

The filter area is selected in such a way that the retentate does not lead to significantly altered filter properties during the filtration process under consideration: the number of retained cells is substantially smaller than the number of pores in the membrane; the projected surface of the retentate (inter alia, retained cells) is substantially smaller than the filter area. The filter membrane is preferably a circular “track etched membrane” made of polycarbonate.

The feed flow is specified by the frequency and the volume of the pipetted blood sample. This need not be constant. The permeate flow need not be constant either. What is crucial for the careful filtration of the CTCs is the least possible mechanical stress on the cells (for example, as a result of shearing forces), which can essentially be realized by means of a small pressure differential. This can be ensured by pressure regulation.

Furthermore, it is possible to filter the respective permeate sequentially using various filter membranes which are distinguished, for example, by size. Provision can be made for the installation of a heating system to enable incubation steps at defined temperatures (for example, for EPISPOT and FISH staining) and the control can compensate for any effects (vapor pressure, etc.) in the process.

Provision is preferably made for level monitoring in the funnel and permeate container by way of corresponding sensors and consideration in the control to prevent drying up or overflowing (adaptive control).

Parallel processing in several filter arrangements is possible as reliable and reproducible filtration properties are ensured. This can take place, for example, by apportioning the volume in the funnel to two or more membranes; pressure in the respective containers can be individually adjusted. In the process, various questions can be investigated at the same time (for example, different pore sizes), the replacement of individual containers is possible without altering the permeate flow. Alternatively, this can take place by apportioning the permeate flow to two or more funnels each with their own filter membrane and container. The parameters (permeate flow as a function of time, pressure, pore size, provision of reagents, etc.) can be individually adjusted. The shared use of resources is advantageous: pressure lines, electrical connections, control and analysis software and the supply of permeate.

Sequential execution is likewise feasible: a connection on the permeate container enables additional filtration; for example by pumping, stacking of the arrangement or a complete filter arrangement inside the permeate container of a first arrangement.

1. A method for filtration of a suspension, comprising:
supplying the suspension to a filter;
determining a pressure differential of pressure upstream and pressure downstream of the filter; and
adjusting a pressure differential upstream compared with downstream of the filter and, as a result, pressing permeate through the filter, wherein the pressure differential does not exceed a determined value.

2. The method of claim 1, wherein the underpressure does not exceed a value of 50 mbar.

3. The method of claim 1, wherein, for determination of the value, pressure through a column of water is also taken into consideration and wherein a 1 cm column of water corresponds to 1 mbar.

4. The method of claim 1, wherein for the supplying of the suspension, a level of a column of water above the filter is monitored and is restricted to a fraction of the pressure differential.

5. The method of claim 1, wherein overpressure is applied upstream of the filter.

6. The method of claim 1, wherein underpressure is applied downstream of the filter.

7. The method of claim 1, wherein the adjusting of the pressure differential is by regulation to a constant value or to a value which is within a range and takes place while permeate is being pressed through the filter.

8. The method of claim 1, wherein the volume of the permeate pressed through the filter is determined.

9. The method of claim 8, wherein the volume of the permeate pressed through the filter is determined by determination of a gas volume supplied when applying overpressure or by determination of a gas volume discharged when applying underpressure.

10. The method of claim 1, wherein the suspension is a suspension of cells in an aqueous solution.

11. The method of claim 1, wherein the filter has a membrane with pores, a direction of the pores running vertically to the surface of the membrane.

12. The method of claim 1, further comprising;
adjusting a pressure differential for a period, the period being selected such that during the period, no permeate is pressed through the membrane.

13. The method of claim 12, wherein the adjusting of the pressure differential comprises adjusting overpressure downstream of the filter for the period, wherein the overpressure is at least as high as the pressure of the column of water above the filter.

14. The method of claim 1, wherein the of a pressure differential is performed by a control.

15. A device, comprising:
a filter;
a liquid reservoir upstream of the filter;
a liquid reservoir downstream of the filter;
a device configured to determine a pressure differential between pressure upstream and pressure downstream of the filter; and
a device configured to adjust a pressure differential upstream compared with downstream of the filter and being configured to press of permeate through the filter, wherein the pressure differential does not exceed a determined value.

16. The device of claim 15, wherein the device configured to determine a pressure differential comprise a differential pressure sensor.

17. The device of claim 15, further comprising:
a device configured to determine the filling level of a liquid above the filter.

18. The device of claim 15, further comprising:
a control, configured to the pressure differential at a constant value or within a range of values while permeate is being pressed through the filter.

19. The device of claim 15, further comprising:
at least one device configured to adjust a pressure differential for a period which is selected such that, during the period, no permeate is pressed through the membrane.

20. The device of claim 19, wherein the at least one device configured to adjust the pressure differential is designed as at least one device configured to adjust overpressure downstream of the filter for a period, wherein the overpressure is at least as high as the pressure of the column of water above the filter.

21. The device of claim 15, further comprising:
at least one device configured to determine the volume of the permeate pressed through the filter.

22. The device of claim 21, wherein the at least one device configured to determine the volume of the permeate pressed through the filter is designed as at least one device configured to determine a gas volume supplied when applying overpressure or as at least one device configured to determine a gas volume discharged when applying underpressure.

23. The device of claim 15, wherein the filter includes a membrane with pores, the direction of the pores running vertically to the surface of the membrane.

24. The device of claim 15, wherein the membrane is a track etched membrane made of polycarbonate.

25. The method as claimed in claim 2, wherein the underpressure does not exceed a value of 10 mbar.

26. The method of claim 2, wherein, for determination of the value, pressure through a column of water is also taken into consideration and wherein a 1 cm column of water corresponds to 1 mbar.

27. The method of claim 25, wherein, for determination of the value, pressure through a column of water is also taken into consideration and wherein a 1 cm column of water corresponds to 1 mbar.

28. The method of claim 4, wherein the fraction is 1/10 of the pressure differential.

29. The method of claim 5, wherein underpressure is applied downstream of the filter.
